(19) United States
(12) Patent Application Publication
(10) Pub. No.: US 2022/0148696 A1
(43) Pub. Date: May 12, 2022

(54) LIFESTYLE HABIT RECOMMENDATION APPARATUS, LIFESTYLE HABIT RECOMMENDATION METHOD, PROGRAM, AND RECORDING MEDIUM

(71) Applicant: NEC Solution Innovators, Ltd., Koto-ku, Tokyo (JP)

(72) Inventors: Jou AKITOMI, Tokyo (JP); Mineko YAMAYUCHI, Tokyo (JP)

(73) Assignee: NEC Solution Innovators, Ltd., Koto-ku, Tokyo (JP)

(21) Appl. No.: 17/435,812

(22) PCT Filed: Mar. 12, 2020
(86) PCT No.: PCT/JP2020/010767

§ 371 Date: Sep. 2, 2021

(30) Foreign Application Priority Data
Mar. 12, 2019 (JP) 2019-045159

(51) Int. Cl. G16H 20/00; A61B 5/00; A61B 5/11

(52) U.S. Cl. CPC G16H 20/00; A61B 5/743; A61B 5/1118; A61B 5/4815

(57) ABSTRACT

An apparatus (10) for lifestyle habit recommendation, comprising at least one processor configured to:
acquire execution information executed for each item serving as an indicator of a lifestyle habit and a vitality level of a sample user in association with a date;
set a scheduled date on which a vitality level of a target user is required to be a predetermined value;
set a given period up to the scheduled date as an output period of recommended lifestyle habit information;
acquire a date on which the associated vitality level of the sample user is the predetermined value, and determines the execution information corresponding to the number of days of the output period up to the date as the recommended information in the output period; and
output the recommended information to the target user.

### TECHNICAL FIELD

The present invention relates to a lifestyle habit recommendation apparatus, a lifestyle habit recommendation method, a program, and a recording medium.

### BACKGROUND ART

Cognitive Behavioral Therapy for Insomnia (CBT-I) is known as an effective treatment modality for insomnia. CBT-I is a psychotherapy that aims to control sleep by reviewing sleep-related cognitive and behavioral habits.

Against this background, an application for recording a sleep habit has been provided (Patent Literature 1). According to this application, it is possible to manage and improve the sleep habit of the user.

### CITATION LIST

### Patent Literature

Patent Literature 1: WO2019/035166

### SUMMARY OF INVENTION

### Technical Problem

Patent Literature 1 discloses the invention that analyzes what has been a hindrance of sleep and generates advice information for eliminating the same, based on data obtained by a user using the application. However, Patent Literature 1 does not recommend an optimal sleep habit for a specific scheduled date.

With the foregoing in mind, it is an object of the present invention to provide a system that recommends an optimal sleep habit for a specific scheduled date.

### Solution to Problem

In order to achieve the above object, the present invention provides a lifestyle habit recommendation apparatus, including: a sample information acquisition unit that acquires execution information executed for each item serving as an indicator of a lifestyle habit and a vitality level of a sample user in association with a date; a scheduled date setting unit that sets a scheduled date on which a vitality level of a target user is required to be a predetermined value; an output period setting unit that sets a given period up to the scheduled date as an output period of recommended lifestyle habit information; a recommended information determination unit that acquires a date on which the associated vitality level of the sample user is the predetermined value, and determines the execution information corresponding to the number of days of the output period up to the date as the recommended information in the output period; and an output unit that outputs the recommended information to the target user.

The present invention also provides a lifestyle habit recommendation method, including the steps of: acquiring sample information by acquiring execution information executed for each item serving as an indicator of a lifestyle habit and a vitality level of a sample user in association with a date; setting a scheduled date on which a vitality level of a target user is required to be a predetermined value; setting an output period by setting a given period up to the scheduled date as an output period of recommended lifestyle habit information; determining recommended information by acquiring a date on which the associated vitality level of the sample user is the predetermined value and determining the execution information corresponding to the number of days of the output period up to the date as the recommended information in the output period; and outputting the recommended information to the target user.

### Advantageous Effects of Invention

The present invention can provide a system that recommends an optimal sleep habit for a specific scheduled date.

### BRIEF DESCRIPTION OF DRAWINGS

FIG. 1 is a block diagram showing a configuration of an example of a lifestyle habit recommendation apparatus according to the first example embodiment.

FIG. 2 is a block diagram showing an example of the hardware configuration of the lifestyle habit recommendation apparatus according to the first example embodiment.

FIGS. 3A and 3B are diagrams showing an example of a screen output on a terminal of a user in the first example embodiment.

FIG. 4 is a flowchart showing an example of the processing in a lifestyle habit recommendation apparatus according to the first example embodiment.

FIG. 5 is a diagram showing an example of determining recommended information based on execution information and a vitality level of a sample user in the first example embodiment.

FIG. 6 is a flowchart showing an example of the processing in a lifestyle habit recommendation apparatus according to the second example embodiment.

### DESCRIPTION OF EXAMPLE EMBODIMENTS

Embodiments of the present invention will be described. Note here that the present invention is not limited to the following example embodiments. In the drawings, identical parts are indicated with identical reference signs. In addition, the descriptions of the respective example embodiments can be referred to each other unless otherwise specified. Furthermore, the configurations of the example embodiments can be combined unless otherwise specified.

In the present invention, the lifestyle habit is not particularly limited, and examples thereof include sleep habits, work habits (overtime, early leaving, etc.), study habits (study hours, study start time, etc.), exercise habits, and eating habits. The lifestyle habit may be, for example, a daily habit or a habit in any other given period.

### First Example Embodiment

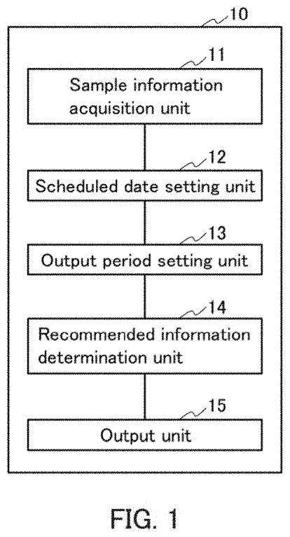

The present example embodiment will be described with reference to a case where the lifestyle habit is a sleep habit and is managed on a daily basis as an example. The present invention, however, is not limited thereto. FIG. 1 is a block diagram showing the configuration of an example of a lifestyle habit recommendation apparatus 1 of the present example embodiment. The lifestyle habit recommendation apparatus 10 includes a sample information acquisition unit 11, a scheduled date setting unit 12, an output period setting unit 13, a recommended information determination unit 14, and an output unit 15. The lifestyle habit recommendation apparatus 10 is also referred to as a lifestyle habit recommendation system, for example.

The lifestyle habit recommendation apparatus 10 may be, for example, a single lifestyle habit recommendation apparatus including the above-described components, or may be a lifestyle habit recommendation apparatus to which the components are connectable via a communication network. The lifestyle habit recommendation apparatus 10 may be, for example, a terminal in which the program of the present invention is installed. Examples of the terminal include a mobile phone, a smartphone, a tablet, and a personal computer (PC). The lifestyle habit recommendation apparatus 10 includes, for example, a terminal and a server, and the terminal and the server may be connectable via a communication network. Examples of the communication network include an Internet line, a telephone line, a local area network (LAN), and a wireless fidelity (WiFi).

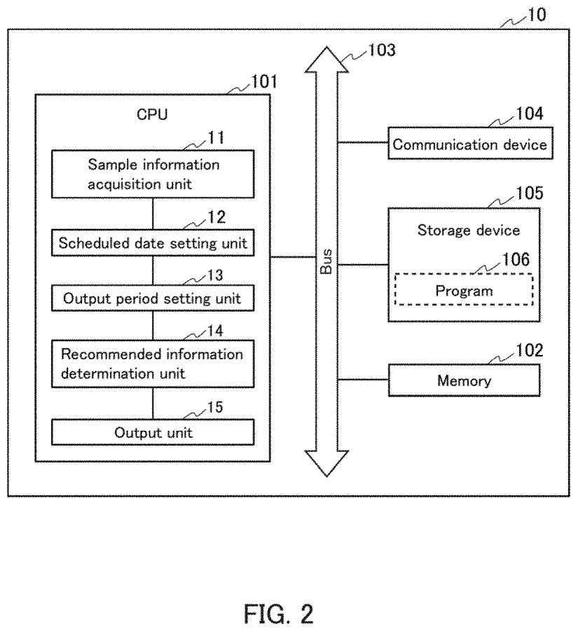

FIG. 2 shows a block diagram of the hardware configuration of the lifestyle habit recommendation apparatus 10. The lifestyle habit recommendation apparatus 10 includes, for example, a central processing unit (CPU) 101, a memory 102, a bus 103, a communication device 104, a storage device 105, and the like. The components of the lifestyle habit recommendation apparatus 10 are connected to each other via a bus 103 by, for example, respective interfaces (I/F).

The CPU 101 serves to control the entire lifestyle habit recommendation apparatus 10. In the lifestyle habit recommendation apparatus 10, the CPU 101 executes a program of the present invention and other programs, and reads and writes various pieces of information, for example. Specifically, for example, the CPU 101 functions as the sample information acquisition unit 11, the scheduled date setting unit 12, the output period setting unit 13, the recommended information determination unit 14, and the output unit 15.

The bus 103 can also be connected to an external device, for example. Examples of the external device include terminals, external storage devices (such as external databases), and printers. The lifestyle habit recommendation apparatus 10 can be connected to a communication network by, for example, a communication device 104 connected to the bus 103, and can also be connected to the external device via the communication network.

The memory 102 includes, for example, a main memory, and the main memory is also referred to as a main storage device. When the CPU 101 performs processing, the memory 102 reads various kinds of operation programs such as the program of the present invention stored in the storage device 105 to be described below, and the CPU 101 receives data from the memory 102 and executes the program. The main memory is, for example, a RAM (random access memory). The memory 102 further includes a ROM (read-only memory), for example.

The storage device 105 is also referred to as a so-called auxiliary storage device with respect to the main memory (main memory device), for example. As described above, the storage device 105 stores operation programs 106 including the program of the present invention. The storage device 105 includes a storage medium and a drive for reading from and writing to the storage medium, for example. The storage medium is not particularly limited, and may be, for example, a built-in type or an external type, and examples thereof include HDs (hard disks), FDs (Floppy® disks), CD-ROMs, CD-Rs, CD-RWs, MOs, DVDs, flash memories, and memory cards. The drive is not particularly limited. The storage device 105 may be a hard disk drive (HDD) in which the storage medium and the drive are integrated, for example. For example, the storage device 105 may store the operation program 106 as described above. Further, the storage device 105 may store, for example, execution information and information such as a vitality level, which will be described below.

The lifestyle habit recommendation apparatus 10 may further include, for example, an input device and an output device such as a display. Examples of the input device include a touch panel, a keyboard, and a mouse. Examples of the display include an LED display and a liquid crystal display.

In the lifestyle habit recommendation apparatus 10, the memory 102 and the storage device 104 may also store access information and log information from the user and information acquired from an external database (not shown).

The sample information acquisition unit 11 acquires execution information executed for each item serving as an indicator of a sleep habit and a vitality level of a sample user in association with a date. The date is, for example, the year, month, and day. The present invention, however, is not limited thereto, and the date may be the number of days elapsed from a given set date or the like.

The sample user is a user whose execution information and vitality level are acquired by the sample information acquisition unit 11 among the users of the lifestyle habit recommendation apparatus 10. On the other hand, the target user to be described below is a user to whom the recommended information is output by the output unit 15 among the users of the lifestyle habit recommendation apparatus 10. The sample user is not particularly limited, and may be, for example, the same user as the target user, users including the target user, or a user(s) different from the target user. The number of sample users may be one or more than one. For example, in the case where the sample user is set as the target user in the default setting and when the sample information acquisition unit 11 cannot sufficiently acquire the execution information and vitality level of the target user, a plurality of users including the target user or a user(s) different from the target user may be used as the sample user.

The items to be the indicators of the sleep habit are not particularly limited, and examples thereof include a record of sleep and supplementary information related to sleep, and specific examples thereof include sleep hours (actual sleep hours), getting-into-bed time (time of getting into bed), falling-asleep-time (time of falling asleep), awaking time (time of awaking), getting-out-of-bed-time (time of getting out of bed), arousal during sleep, sleep efficiency (ratio of sleep hours to bedtime), behavior before sleeping, food and drink (alcohol, coffee, tobacco, etc.), and whether or not to take a nap and its time.

The vitality level is a value indicating the vitality level of the user, and is not particularly limited, and, for example, may be based on the subjectivity of the user, may be a value (a biological value or the like) measured by a sensor or the like, or may be a result of a given test for measuring the vitality level. The vitality level can also be referred to as, for example, a vigor level, a performance, and the like.

The execution information and the vitality level may be acquired based on input by the user or may be acquired directly from the sensor or the like, for example. The sample information acquisition unit 11 may calculate the execution information based on the acquired data, for example. As the execution information and the vitality level data, for example, the accumulated data can be used by the user managing the sleep habit using the lifestyle habit recommendation apparatus 10.

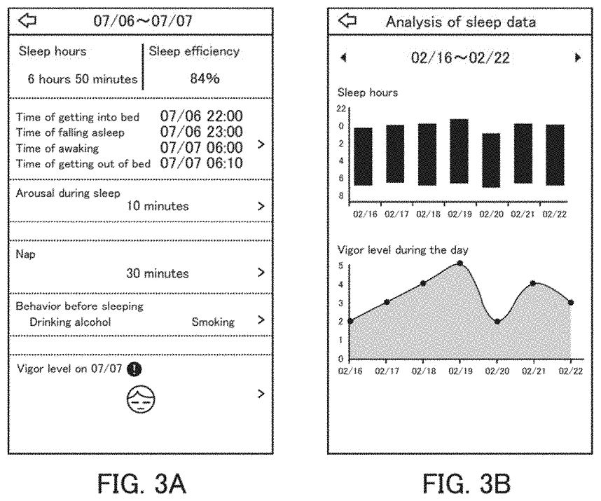

FIGS. 3A and 3B show an example of a display screen of a terminal of the user. In FIG. 3A, based on the input to the terminal by the user, as the execution information of July 6 (to July 7), the sleep hours, the sleep efficiency, the time of getting into bed, the time of falling asleep, the time of awaking, the time of getting out of bed, the arousal during sleep, the nap hours, and the food and drink taken before sleeping are displayed, and as the vitality level on July 7, the vigor level during the day is displayed (in FIG. 3A, the vigor level is scored in five stages and displayed by the expression of the character). In FIG. 3B, the sleep hours is displayed as the execution information from February 16 to February 22, and the vigor level during the day is displayed as the vitality level.

The scheduled date setting unit 12 sets a scheduled date on which the vitality level of the target user is required to be a predetermined value. The scheduled date is not particularly limited, and can be set based on, for example, input by the target user. Examples of the scheduled date include the day of the test, the day of the sport match, the day of the presentation, and the day to go to the trip. The predetermined value is, for example, a value of high vitality level. The predetermined value may be, for example, just one value or a value having a range.

The output period setting unit 13 sets a given period up to the scheduled date as an output period of recommended lifestyle habit information. The given period is not particularly limited, and may be, for example, a period from a date set by the target user to the scheduled date, and may be, for example, 1 to 7 days. The date set by the target user may be the date set based on the input by the target user or may be the date on which the scheduled date is set by the scheduled date setting unit 12, or the like.

The recommended information determination unit 14 acquires a date on which the associated vitality level of the sample user is the predetermined value, and determines the execution information corresponding to the number of days of the output period up to the date as the recommended information in the output period.

The output unit 15 outputs the recommended information to the target user. The recommended information output may be transmitted to the terminal of the target user via the communication device 104, may be displayed on the display or the like of the lifestyle habit recommendation apparatus 10, or may be output to a file, for example.

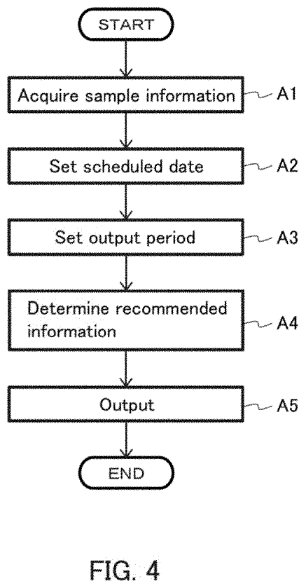

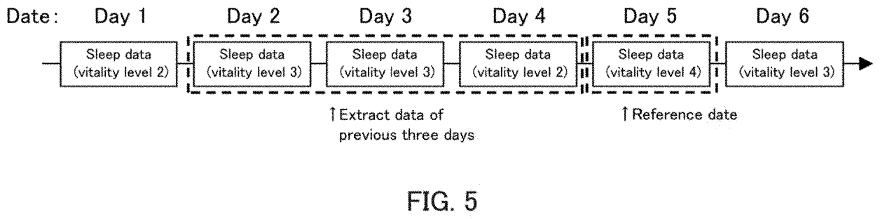

Next, the lifestyle habit recommendation method of the present example embodiment will be described with reference to FIGS. 4 and 5. FIG. 4 is a flowchart showing an example of the lifestyle habit recommendation method. FIG. 5 is a diagram showing an example of determining the recommended information based on the execution information and the vitality level of the sample user. The lifestyle habit recommendation method of the present example embodiment can be implemented as follows using, for example, the lifestyle habit recommendation apparatus 10 of FIG. 1. The lifestyle habit recommendation method of the present example embodiment is not limited to the use of the lifestyle habit recommendation apparatus 10 of FIG. 1.

First, the sample information acquisition unit 11 acquires execution information executed for each item serving as an indicator of the sleep habit and a vitality level of a sample user in association with a date (step (A1)). For example, as shown in FIG. 5, the execution information (sleep data) and the vitality level of the sample user are acquired on each date of day 1 to day 6.

Next, the scheduled date setting unit 12 sets a scheduled date on which the vitality level of the target user is required to be a predetermined value (step (A2)). In the example of FIG. 5, the predetermined value is required to be “4 or more” on the scheduled date (not shown).

Next, the output period setting unit 13 sets a given period up to the scheduled date as an output period of recommended sleep habit information (step (A3)). In the example of FIG. 5, three days up to the scheduled date are set as the output period.

Next, the recommended information determination unit 14 acquires a date on which the associated vitality level of the sample user is the predetermined value, and determines the execution information corresponding to the number of days of the output period up to the date as the recommended information in the output period (step (A4)). In the example of FIG. 5, the date (reference date) on which the vitality level of the sample user is “4 or more” is “day 5”. Therefore, the execution information (sleep data) for 3 days up to the “day 5” is determined as the recommended information in the output period.

Next, the output unit 15 outputs the recommended information to the target user (step (A5)), and ends the procedure (END).

In this manner, according to the present example embodiment, past execution information of a user is output as recommended sleep habit information up to the scheduled date on which the vitality level is required to be a predetermined value. Thereby, for example, sleep habits can be managed so as to increase the performance (vitality level) towards future destination dates. The present invention can also be applied to, for example, prevention of j et lag.

### Second Example Embodiment

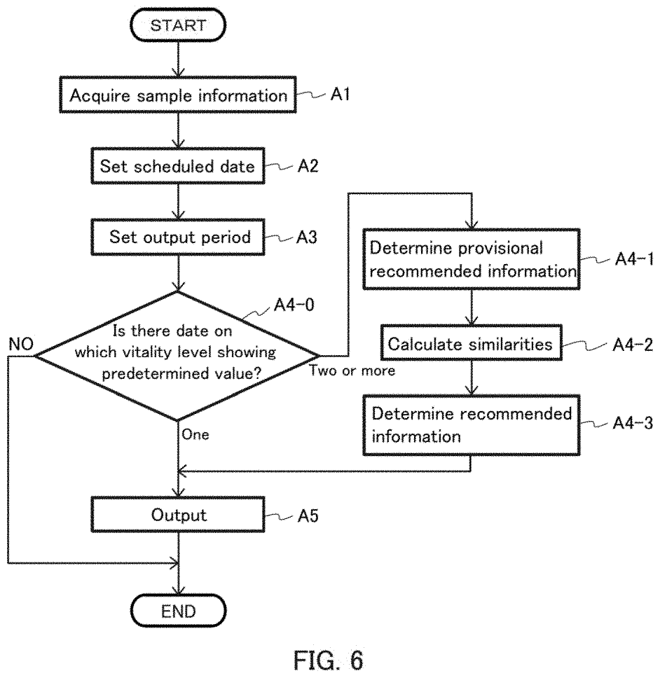

Next, the second example embodiment will be described with reference to FIG. 6. FIG. 6 is a flowchart showing an example of the lifestyle habit recommendation method. The lifestyle habit recommendation method according to the present example embodiment relates to a case where the recommended information determination unit 14 acquires a plurality of the dates on which the vitality level of the sample user is the predetermined value in the step (A4). The second example embodiment is the same as the first example embodiment other than this point.

That is, in the step (A4), first, when the recommended information determination unit 14 acquires a plurality of the dates on which the associated vitality level of the sample user is the predetermined value (step (A4-0)), the recommended information determination unit 14 uses the execution information corresponding to the number of days of the output period up to each of the plurality of the dates as provisional recommended information in the output period (step (A4-1)). On the other hand, when the recommended information determination unit 14 acquires one date on which the associated vitality level of the sample user is the predetermined value in the step (A4-0), the process proceeds to the step (A5). When the recommended information determination unit 14 does not acquire the date on which the associated vitality level of the sample user is the predetermined value in the step (A4-0), the procedure is ended (END).

Next, the recommended information determination unit 14 determines whether or not similarities of the provisional recommended information exceed a predetermined threshold (step (A4-2)).

The determination can be performed by a known method, for example, as follows. That is, the items serving as indicators of the sleep habits in the provisional recommended information are converted into feature vectors, respectively. The conversion into the feature vector is not particularly limited, and for example, the time and the time period may be converted into 0.25 every 15 minutes (for example, “6.5” for “6:30”), and the case where there is an action may be converted into 1 and the case where there is no action may be converted into 0. Next, as the similarity of the provisional recommended information, a correlation, a cosine similarity, or the like between the feature vectors is calculated. Then, it is determined whether or not the similarity exceeds a predetermined threshold. The predetermined threshold value is not particularly limited and can be set freely.

Next, the recommended information determination unit 14 determines the provisional recommended information having the largest number of similarities determined to exceed the predetermined threshold as the recommended information (step (A4-3)), and proceeds to the step (A5).

The present example embodiment can output more reliable recommended information in addition to the effect of the first example embodiment, for example.

(Variation)

Next, a variation of the second example embodiment will be described. In the lifestyle habit recommendation method according to the present variation, the sample user includes the target user. The present variation is the same as the second example embodiment other than this point.

After the step (A4-0), the recommended information determination unit 14 determines the provisional recommended information based on the execution information of the target user and the provisional recommended information based on the execution information of the sample user other than the target user in the step (A4-1).

Next, the recommended information determination unit 14 determines whether or not similarities between the provisional recommended information based on the execution information of the target user and the provisional recommended information based on the execution information of the sample user other than the target user exceed a predetermined threshold in the step (A4-2).

Next, the recommended information determination unit 14 determines, among the provisional recommended information based on the execution information of the target user, the provisional recommended information having the largest number of similarities determined to exceed the predetermined threshold as the recommended information in the step (A4-3), and proceeds to the step (A5).

In the step (A4-3), when none of the provisional recommended information based on the execution information of the target user has similarities determined to exceed the predetermined threshold, the provisional recommended information having the largest number of similarities determined to exceed the predetermined threshold, among the provisional recommended information based on the execution information of the sample user other than the target user, may be determined as the recommended information, for example.

The present variation can output the recommended information based on the sleep habit data of the target user in addition to the effects of the first and second example embodiments, for example.

### Third Example Embodiment

The program of the present example embodiment is a program for a computer to execute the lifestyle habit recommendation method of each of the example embodiments. The program of the present example embodiment may be recorded on, for example, a computer readable recording medium. The recording medium is not particularly limited, and examples thereof include read-only memories (ROMs), hard disks (HDs), optical disks, and Floppy® disks (FDs).

While the present invention has been described above with reference to illustrative example embodiments, the present invention is by no means limited thereto. Various changes and variations that may become apparent to those skilled in the art may be made in the configuration and specifics of the present invention without departing from the scope of the present invention.

This application claims priority from Japanese Patent Application No. 2019-045159 filed on Mar. 12, 2019. The entire subject matter of the Japanese Patent Applications is incorporated herein by reference.

### INDUSTRIAL APPLICABILITY

The present invention can provide a system that recommends an optimal sleep habit for a specific scheduled date.

(Supplementary Notes)

Some or all of the above example embodiments may be described as in the following Supplementary Notes, but are not limited thereto.

(Supplementary Note 1)

A lifestyle habit recommendation apparatus, including:

a sample information acquisition unit that acquires execution information executed for each item serving as an indicator of a lifestyle habit and a vitality level of a sample user in association with a date;

a scheduled date setting unit that sets a scheduled date on which a vitality level of a target user is required to be a predetermined value;

an output period setting unit that sets a given period up to the scheduled date as an output period of recommended lifestyle habit information;

a recommended information determination unit that acquires a date on which the associated vitality level of the sample user is the predetermined value, and determines the execution information corresponding to the number of days of the output period up to the date as the recommended information in the output period; and

an output unit that outputs the recommended information to the target user.

(Supplementary Note 2)

The lifestyle habit recommendation apparatus according to Supplementary Note 1, wherein

the sample user includes the target user.

(Supplementary Note 3)

The lifestyle habit recommendation apparatus according to Supplementary Note 1 or 2, wherein

the given period up to the scheduled date is a period from a date set by the target user to the scheduled date.

(Supplementary Note 4)

The lifestyle habit recommendation apparatus according to any one of Supplementary Notes 1 to 3, wherein

when the recommended information determination unit acquires a plurality of the dates on which the associated vitality level of the sample user is the predetermined value,

the recommended information determination unit

uses the execution information corresponding to the number of days of the output period up to each of the plurality of the dates as provisional recommended information in the output period,

determines whether or not similarities among the provisional recommended information exceed a predetermined threshold, and

determines the provisional recommended information having the largest number of similarities determined to exceed the predetermined threshold as the recommended information.

(Supplementary Note 5)

The lifestyle habit recommendation apparatus according to Supplementary Note 4, wherein

the sample user includes the target user,

the recommended information determination unit

determines whether or not similarities between the provisional recommended information based on the execution information of the target user and the provisional recommended information based on the execution information of the sample user other than the target user exceed a predetermined threshold, and

determines, among the provisional recommended information based on the execution information of the target user, the provisional recommended information having the largest number of similarities determined to exceed the predetermined threshold as the recommended information.

(Supplementary Note 6)

The lifestyle habit recommendation apparatus according to any one of Supplementary Notes 1 to 5, wherein

the lifestyle habit is a sleep habit.

(Supplementary Note 7)

A lifestyle habit recommendation method, including the steps of:

acquiring sample information by acquiring execution information executed for each item serving as an indicator of a lifestyle habit and a vitality level of a sample user in association with a date;

setting a scheduled date on which a vitality level of a target user is required to be a predetermined value;

setting an output period by setting a given period up to the scheduled date as an output period of recommended lifestyle habit information;

determining recommended information by acquiring a date on which the associated vitality level of the sample user is the predetermined value and determining the execution information corresponding to the number of days of the output period up to the date as the recommended information in the output period; and

outputting the recommended information to the target user.

(Supplementary Note 8)

The lifestyle habit recommendation method according to Supplementary Note 7, wherein

the sample user includes the target user.

(Supplementary Note 9)

The lifestyle habit recommendation method according to Supplementary Note 7 or 8, wherein

the given period up to the scheduled date is a period from a date set by the target user to the scheduled date.

(Supplementary Note 10)

The lifestyle habit recommendation method according to any one of Supplementary Notes 7 to 9, wherein

when the recommended information-determining acquires a plurality of the dates on which the associated vitality level of the sample user is the predetermined value,

the recommended information-determining

uses the execution information corresponding to the number of days of the output period up to each of the plurality of the dates as provisional recommended information in the output period,

determines whether or not similarities among the provisional recommended information exceed a predetermined threshold, and

determines the provisional recommended information having the largest number of similarities determined to exceed the predetermined threshold as the recommended information.

(Supplementary Note 11)

The lifestyle habit recommendation method according to Supplementary Note 10, wherein

the sample user includes the target user,

the recommended information-determining

determines whether or not similarities between the provisional recommended information based on the execution information of the target user and the provisional recommended information based on the execution information of the sample user other than the target user exceed a predetermined threshold, and

determines, among the provisional recommended information based on the execution information of the target user, the provisional recommended information having the largest number of similarities determined to exceed the predetermined threshold as the recommended information.

(Supplementary Note 12)

The lifestyle habit recommendation method according to any one of Supplementary Notes 7 to 11, wherein

the lifestyle habit is a sleep habit.

(Supplementary Note 13)

A program for a computer to execute the method according to any one of Supplementary Notes 7 to 12.

(Supplementary Note 14)

A computer readable recording medium with the program according to Supplementary Note 13.

### REFERENCE SIGNS LIST

10: lifestyle habit recommendation apparatus
11: sample information acquisition unit
12: scheduled date setting unit
13: output period setting unit
14: recommended information determination unit
15: output unit

What is claimed is:

1. An apparatus for lifestyle habit recommendation, comprising at least one processor configured to:
acquire execution information executed for each item serving as an indicator of a lifestyle habit and a vitality level of a sample user in association with a date;
set a scheduled date on which a vitality level of a target user is required to be a predetermined value;
set a given period up to the scheduled date as an output period of recommended lifestyle habit information;
acquire a date on which the associated vitality level of the sample user is the predetermined value, and determines the execution information corresponding to the number of days of the output period up to the date as the recommended information in the output period; and
output the recommended information to the target user.

2. The apparatus according to claim 1, wherein
the sample user includes the target user.

3. The apparatus according to claim 1, wherein
the given period up to the scheduled date is a period from a date set by the target user to the scheduled date.

4. The apparatus according to claim 1, wherein
when the processor acquires a plurality of the dates on which the associated vitality level of the sample user is the predetermined value,
the processor is configured to use the execution information corresponding to the number of days of the output period up to each of the plurality of the dates as provisional recommended information in the output period;
the processor is configured to determine whether or not similarities among the provisional recommended information exceed a predetermined threshold;
the processor is configured to determine the provisional recommended information having the largest number of similarities determined to exceed the predetermined threshold as the recommended information.

5. The apparatus according to claim 4, wherein
the sample user includes the target user,
the processor is configured to determine whether or not similarities between the provisional recommended information based on the execution information of the target user and the provisional recommended information based on the execution information of the sample user other than the target user exceed a predetermined threshold, and
, the processor is configured to determine among the provisional recommended information based on the execution information of the target user, the provisional recommended information having the largest number of similarities determined to exceed the predetermined threshold as the recommended information.

6. The apparatus according to claim 1, wherein
the lifestyle habit is a sleep habit.

7. A computer-implemented method for a lifestyle habit recommendation, comprising:
acquiring sample information by acquiring execution information executed for each item serving as an indicator of a lifestyle habit and a vitality level of a sample user in association with a date;
setting a scheduled date on which a vitality level of a target user is required to be a predetermined value;
setting an output period by setting a given period up to the scheduled date as an output period of recommended lifestyle habit information;
determining recommended information by acquiring a date on which the associated vitality level of the sample user is the predetermined value and determining the execution information corresponding to the number of days of the output period up to the date as the recommended information in the output period; and
outputting the recommended information to the target user.

8. The method according to claim 7, wherein
the sample user includes the target user.

9. The method according to claim 7, wherein
the given period up to the scheduled date is a period from a date set by the target user to the scheduled date.

10. The method according to claim 7 comprising:
if acquiring a plurality of the dates on which the associated vitality level of the sample user is the predetermined value,
using the execution information corresponding to the number of days of the output period up to each of the plurality of the dates as provisional recommended information in the output period,
determining whether or not similarities among the provisional recommended information exceed a predetermined threshold, and
determining the provisional recommended information having the largest number of similarities determined to exceed the predetermined threshold as the recommended information.

11. The method according to claim 10, wherein
the sample user includes the target user, wherein
the method comprising:
determining whether or not similarities between the provisional recommended information based on the execution information of the target user and the provisional recommended information based on the execution information of the sample user other than the target user exceed a predetermined threshold;
determining, among the provisional recommended information based on the execution information of the target user, the provisional recommended information having the largest number of similarities determined to exceed the predetermined threshold as the recommended information.

12. The lifestyle habit recommendation method according to claim 7, wherein
the lifestyle habit is a sleep habit.

13. (canceled)

14. A non-transitory computer readable recording medium with a program, wherein
the program cause a computer execute a method for a lifestyle habit recommendation, wherein,
the method comprising:
acquiring sample information by acquiring execution information executed for each item serving as an indicator of a lifestyle habit and a vitality level of a sample user in association with a date;
setting a scheduled date on which a vitality level of a target user is required to be a predetermined value;
setting an output period by setting a given period up to the scheduled date as an output period of recommended lifestyle habit information;
determining recommended information by acquiring a date on which the associated vitality level of the sample user is the predetermined value and determining the execution information corresponding to the number of days of the output period up to the date as the recommended information in the output period; and
outputting the recommended information to the target user.

15. The non-transitory computer readable recording medium according to claim 14, wherein
the sample user includes the target user.

16. The non-transitory computer readable recording medium according to claim 14, wherein
the given period up to the scheduled date is a period from a date set by the target user to the scheduled date.

17. The non-transitory computer readable recording medium according to claim 14, wherein
the method comprising:
if acquiring a plurality of the dates on which the associated vitality level of the sample user is the predetermined value, using the execution information corresponding to the number of days of the output period up to each of the plurality of the dates as provisional recommended information in the output period;
determining whether or not similarities among the provisional recommended information exceed a predetermined threshold;
determining the provisional recommended information having the largest number of similarities determined to exceed the predetermined threshold as the recommended information.

18. The non-transitory computer readable recording medium according to claim 17, wherein
the sample user includes the target user, wherein
the method comprising:
determining whether or not similarities between the provisional recommended information based on the execution information of the target user and the provisional recommended information based on the execution information of the sample user other than the target user exceed a predetermined threshold;
determining, among the provisional recommended information based on the execution information of the target user, the provisional recommended information having the largest number of similarities determined to exceed the predetermined threshold as the recommended information.

19. The non-transitory computer readable recording medium according to claim 14, wherein
the lifestyle habit is a sleep habit.
